(19) United States
(12) Patent Application Publication
(10) Pub. No.: US 2014/0275138 A1
(43) Pub. Date: Sep. 18, 2014

(54) METHOD AND PRODUCTS FOR TREATING DIABETES

(71) Applicant: CBA Pharma, Inc., Lexington, KY (US)

(72) Inventor: James A. Mitchell, Grand Rapids, MI (US)

(73) Assignee: CBA Pharma, Inc., Lexington, KY (US)

(21) Appl. No.: 14/204,545

(22) Filed: Mar. 11, 2014

Related U.S. Application Data
(60) Provisional application No. 61/798,713, filed on Mar. 15, 2013.

(51) Int. Cl. A61K 31/4748; A61K 45/06

(52) U.S. Cl. CPC A61K 31/4748; A61K 45/06
USPC 514/279

(57) ABSTRACT

Treating diabetes by administering one or more members of the d-tetrandrine family, either alone or with other Type 2 diabetes drugs or dietary supplements which mediate diabetes.

### CROSS REFERENCE TO RELATED APPLICATIONS

The present application claims the benefit of U.S. Provisional Patent Application No. 61/798,713, entitled METHOD AND PRODUCTS FOR TREATING DIABETES, filed on Mar. 15, 2013, the entire contents of which are incorporated by reference.

### FIELD AND BACKGROUND OF THE INVENTION

The present invention relates to the treatment of Type 2 diabetes. Type 2 diabetes is usually caused by an increase in insulin resistance, wherein insulin interaction with its cell receptor is either interfered with, or fails to elicit the normal downstream reactions which lower blood glucose levels. Type 2 diabetes is treated with drugs which

(1) stimulate the pancreas to produce and release more insulin,

(2) inhibit the production and release of glucose from the liver,

(3) blocking the action of stomach enzymes that break down carbohydrates, or

(4) improving the sensitivity of cells to insulin.

### SUMMARY OF THE INVENTION

In the present invention, diabetes is treated by the administration of particular members of the d-tetrandrine family of drugs. The d-tetrandrine family members have the following structural formula:

[chemical structure]

Where R₁ and R₁′ are the same or different shortchained carbon based ligand including without limitation, CH₃, CO₂CH₃ or H; and R₂ is CH₃ or C₂H₅; and R₃ is CH₃ or hydrogen; and where the chemical structure has the “S” isomeric configuration at the C-1′ chiral carbon location. The treatment may involve treatment with the d-tetrandrine family member only, or in conjunction with other drugs used to treat diabetes.

### DESCRIPTION OF THE PREFERRED EMBODIMENT

The preferred members of the d-tetrandrine family include the following representative examples, which are not intended to be exhaustive: d-tetrandrine, isotetrandrine, hernandezine, berbamine, pycnamine, phaeanthine, obamegine, ethyl fangchinoline and fangchinoline. In all of these examples, R₁ and R₁′ constitute the methyl group. Variation within the group occurs in that R₂ and R₃ may constitute either a methyl group or hydrogen, and the isometric configuration of the compounds at the C-1 and C-1′ chiral carbon positions is either R (rectus) or S (sinister). The rules for R and S configuration can be bound in Morrison and Boyd, “Organic Chemistry,” 4^{th} Edition, copyright 1983 by Allyn and Bacon, at pp. 138-141. As noted above, the chiral configuration at C-1 is “S” for members of the d-tetrandrine family. In addition, hernandezine includes a methoxy group at the C-5 position.

The most preferred member of the claimed d-tetrandrine family is d-tetrandrine. Methods for extracting and/or purifying d-tetrandrine are disclosed in U.S. Pat. No. 6,218,541 and in Published Patent Application No. 2011/0105755.

While not wishing to be bound by a particular theory on mode of operation, it is believed that the d-tetrandrine family member acts to improve the sensitivity of cells to insulin. As such, the d-tetrandrine family member can be administered alone as a Type 2 diabetes treatment.

However, the d-tetrandrine family member can also be administered concurrently with a secondary Type 2 diabetes medication. The most preferred drug to be administered concurrently would be one of the drugs which act to improve the sensitivity of cells to insulin. Similarly, there are dietary supplements which mediate diabetes. The d-tetrandrine family member can also be administered concurrently with such dietary supplements.

In the case of concurrent administration, the d-tetrandrine family member and the secondary diabetes drug or dietary supplement can be formulated together into a single formula, or they can be formulated separately and administered either simultaneously or sufficiently close together that they are both in the target cell area at the same time. The d-tetrandrine family member and the diabetes drug or dietary supplement can be formulated separately and be sold as part of a “kit.” The usage ratio of the d-tetrandrine family member to a diabetes drug or dietary supplement will vary from patient to patient and as a function of the secondary drug or dietary supplement used, within a range of about 10:90 to 90:10, but more preferably from about 25:75 to 75:25.

It is believed that the optimum dosage procedure would be to administer the d-tetrandrine family member in oral doses of from about 50 to about 1000 mg per square meter per day, more preferably 250-700, and most preferably about 500, (probably in two to four doses per day). For concurrent administration, a secondary diabetes drug or dietary supplement would preferably be administered simultaneously or on the same day. The dosage level for the d-tetrandrine family member will vary from case to case, based on the patient and on the drug or dietary supplement used. The drug or dietary supplement is administered at usual dosage levels (possibly somewhat less in view of the effect of the tetrandrine family member) once or more during the course of the d-tetrandrine family member dosing.

The d-tetrandrine family bisbenzylisoquinolines have two nitrogen locations and hence can exist in the free base form or as a mono or di-acid salt. Because of the enhanced solubility of the salt form of pharmaceutical ingredients, the salt forms are used in formulating pharmaceutical compositions. The active ingredient thus solubilizes more quickly and enters the bloodstream faster. The free base form is not soluble in water. However, it has recently been surprisingly found by a co-worker that the free base formulations of d-tetrandrine family members are absorbed into the bloodstream substantially as rapidly as formulations of the di-acid salt members of the family. Accordingly, we propose to use either the free base or the di-acid salt of the d-tetrandrine family member in our formulations.

The preferred formulations comprise a member of the d-tetrandrine family combined with a suitable pharmaceutical carrier. The pharmaceutical carrier can be a liquid or a solid composition. A liquid carrier will preferably comprise water, possibly with additional ingredients such as 0.25% carboxymethylcellulose. The solid carrier or diluent used may be pregelatinized starch, microcrystalline cellulose or the like. It may also be formulated with other ingredients, such as colloidal silicone dioxide, sodium lauryl sulfate and magnesium stearate.

A 200 mg capsule, tablet or liquid dosage formulation is most preferred. The most preferred dose of about 500 mg/square meter/day is roughly 1000 mg per day for a 190 pound patient six feet tall. Such a patient can fulfill the dosage requirements by taking five capsules during the course of the day, for example three in the morning and two in the evening, or one at a time spaced out over the day. A smaller person weighing 125 pounds at a height of five feet six inches would require four 200 mg capsules during the course of the day.

Of course, it is understood that the forgoing are preferred embodiments of the invention, and that variations can be employed without departing from the spirit of the invention as set forth in the appended claims, interpreted in accordance with the principles of patent law.

1. A method of treating Type 2 diabetes comprising:
administering to a patient with Type 2 diabetes a member of the d-tetrandrine family of drugs having the following structural formula: [chemical structure]
where R₁ and R₁′ are the same or different short chained carbon based ligand including without limitation, CH₃, CO₂CH₃ or H; and R₂ is CH₃ or C₂H₅; and R₃ is CH₃ or hydrogen, and wherein said structural formula has the “S” isomeric configuration at the C-1′ chiral carbon location.

2. The method of claim 1 wherein said member of the d-tetrandrine family is selected from the group consisting of: d-tetrandrine, isotetrandrine, hernandezine, berbamine, pycnamine, phaeanthine, obamegine, ethyl fangchinoline and fangchinoline.

3. The method of claim 1 wherein said member of the d-tetrandrine family is d-tetrandrine.

4. The method of claim 3 in which the d-tetrandrine family member is used in conjunction with an additional Type 2 diabetes medication which sensitizes cells to insulin, and/or a dietary supplement which mediates diabetes.

5. The method of claim 4 in which the d-tetrandrine family member and said additional Type 2 diabetes medication and/or dietary supplement which mediates diabetes, are formulated together into a single formulation.

6. The method of claim 1 in which the d-tetrandrine family member is used in conjunction with an additional Type 2 diabetes medication, and/or dietary supplement which mediates diabetes.

7. The method of claim 6 in which the d-tetrandrine family member and the additional Type 2 diabetes medication and/or dietary supplement which mediates diabetes are formulated together into a single formulation.

8. The method of claim 6 in which the d-tetrandrine family member and the additional Type 2 diabetes medication and/or dietary supplement which mediates diabetes are formulated separately and administered either simultaneously or sufficiently close together that the insulin receptors are exposed to both simultaneously.

9. The method of claim 8 wherein said member of the d-tetrandrine family is d-tetrandrine.

10. The method of claim 6 in which the d-tetrandrine family member and the additional Type 2 diabetes medication and/or dietary supplement which mediates diabetes are administered in a usage ratio of d-tetrandrine family member to drug or supplement, within a range of from about 0.04 to about 170.

11. The method of claim 6 in which the d-tetrandrine family member and the additional Type 2 diabetes medication and/or dietary supplement which mediates diabetes are administered in a usage ratio of d-tetrandrine family member to drug or dietary supplement, within a range of from about 1 to 100.

12. The method of claim 6 in which the d-tetrandrine family member and the additional Type 2 diabetes medication and/or dietary supplement which mediates diabetes are administered in a usage ratio of d-tetrandrine family member to drug or dietary supplement, within a range of from about 25:75 to 75:25.

13. The method of claim 6 in which the d-tetrandrine family is administered in oral doses of from about 50 to about 1000 mg per square meter per day over a period of from about 4 to about 14 days, and the additional Type 2 diabetes medication and/or dietary supplement which mediates diabetes is then administered at usual dosage levels once or more during said 4 to 14 days.

14. The method of claim 13 in which the d-tetrandrine family is administered in oral doses of from about 250-700 mg per square meter per day over said period of from about 4 to about 14 days.

15. The method of claim 13 in which the d-tetrandrine family is administered in oral doses of about 500 mg per square meter per day over said period of from about 4 to about 14 days, in two to four doses per day.

16. The method of claim 1 in which the d-tetrandrine family is administered in oral doses of from about 50 to about 1000 mg per square meter per day over a period of from about 4 to about 14 days.

17. The method of claim 1 in which the d-tetrandrine family is administered in oral doses of from about 250-700 mg per square meter per day over said period of from about 4 to about 14 days.

18. The method of claim 1 in which the d-tetrandrine family is administered in oral doses of about 500 mg per square meter per day over said period of from about 4 to about 14 days, in two to four doses per day.

19. A pharmaceutical composition for treating Type 2 diabetes, comprising a Type 2 diabetes medication and/or dietary supplement which mediates diabetes, combined with a member of the d-tetrandrine family having the following structural formula: [chemical structure]
where R₁ and R₁′ are the same or different short chained carbon based ligand including without limitation, CH₃, CO₂CH₃ or H; and R₂ is CH₃ or C₂H₅; and R₃ is CH₃ or hydrogen, and wherein said structural formula has the “S” isomeric configuration at the C-1′ chiral carbon location.

20. A pharmaceutical kit for treating Type 2 diabetes, including a Type 2 diabetes medication and/or dietary supplement which mediates diabetes, and a formulation comprising a member of the d-tetrandrine family having the following structural formula: [chemical structure]
where R₁ and R₁′ are the same or different short chained carbon based ligand including without limitation, CH₃, CO₂CH₃ or H; and R₂ is CH₃ or C₂H₅; and R₃ is CH₃ or hydrogen, and wherein said structural formula has the “S” isomeric configuration at the C-1′ chiral carbon location.
